(19) United States
(12) Patent Application Publication
(10) Pub. No.: US 2018/0021088 A1
(43) Pub. Date: Jan. 25, 2018

(54) OPTIC FIBER FIXTURES FOR ILLUMINATED LASER PROBES

(71) Applicant: Katalyst Surgical, LLC, Chesterfield, MO (US)

(72) Inventors: Gregg D. Scheller, Wildwood, MO (US); Matthew N. Yates, High Ridge, MO (US); Steven G. Scheller, Chesterfield, MO (US); Daniel J. Wiener, St. Charles, MO (US)

(73) Assignee: Katalyst Surgical, LLC, Chesterfield, MO (US)

(21) Appl. No.: 15/640,663

(22) Filed: Jul. 3, 2017

Related U.S. Application Data
(60) Provisional application No. 62/364,086, filed on Jul. 19, 2016.

(51) Int. Cl. A61B 18/22; A61F 9/008

(52) U.S. Cl. CPC A61B 18/22; A61F 9/008; A61B 2018/2005

(57) ABSTRACT

A single-use illuminated laser probe may include an optic fiber, an illumination optic fiber, a handle, a hypodermic tube, a transitory connector, and an illumination optic fiber housing. The optic fiber may be disposed in the hypodermic tube, the handle, and the transitory connector. The illumination optic fiber may be disposed in the hypodermic tube, the handle, and the illumination optic fiber housing. A user may insert the transitory connector in an optic fiber fixture to transmit laser light through the optic fiber. A user may insert the illumination optic fiber housing in an illumination optic fiber fixtures to transmit illumination light through the illumination optic fiber.

### CROSS-REFERENCE TO RELATED APPLICATIONS

This application claims the benefit of U.S. Provisional Application No. 62/364,086, filed Jul. 19, 2016.

### FIELD OF THE INVENTION

The present disclosure relates to a surgical instrument, and, more particularly, to optic fiber fixtures for illuminated laser probes.

### BACKGROUND OF THE INVENTION

A wide variety of ophthalmic procedures require a laser energy source. For example, ophthalmic surgeons may use laser photocoagulation to treat proliferative retinopathy. Proliferative retinopathy is a condition characterized by the development of abnormal blood vessels in the retina that grow into the vitreous humor. Ophthalmic surgeons may treat this condition by energizing a laser to cauterize portions of the retina to prevent the abnormal blood vessels from growing and hemorrhaging. Typically, treatments are performed using a disposable, single-use laser probe connected to a laser surgical machine by an optical fiber. Unfortunately, use of disposable, single-use laser probes increases treatment costs because a new laser probe is required for each surgical treatment. Accordingly, there is a need for a laser probe that may be safely used to perform more than one surgical procedure.

### BRIEF SUMMARY OF THE INVENTION

The present disclosure provides optic fiber fixtures for illuminated laser probes. Illustratively, a single-use illuminated laser probe may comprise an optic fiber, an illumination optic fiber, a handle, a hypodermic tube, a transitory connector, and an illumination optic fiber housing. In one or more embodiments, the optic fiber may be disposed in the hypodermic tube, the handle, and the transitory connector. Illustratively, the illumination optic fiber may be disposed in the hypodermic tube, the handle, and the illumination optic fiber housing. In one or more embodiments, a user may insert the transitory connector in an optic fiber fixture to transmit laser light through the optic fiber. Illustratively, a user may insert the illumination optic fiber housing in an illumination optic fiber fixtures to transmit illumination light through the illumination optic fiber.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A and 1B are schematic diagrams illustrating a transitory connector;

FIG. 2 is a schematic diagram illustrating an exploded view of a single-use laser probe assembly;

FIGS. 3A and 3B are schematic diagrams illustrating an assembled single-use laser probe;

FIG. 4 is a schematic diagram illustrating an exploded view of an optic fiber fixture assembly;

FIGS. 5A and 5B are schematic diagrams illustrating an assembled optic fiber fixture;

FIGS. 6A and 6B are schematic diagrams illustrating a single-use laser probe with optic fiber fixture;

FIG. 7 is a schematic diagram illustrating an exploded view of a single-use illuminated laser probe assembly;

FIGS. 8A and 8B are schematic diagrams illustrating an assembled single-use illuminated laser probe;

FIGS. 9A and 9B are schematic diagrams illustrating an illumination optic fiber fixture;

FIGS. 10A and 10B are schematic diagrams illustrating a single-use illuminated laser probe with optic fiber fixtures.

### DETAILED DESCRIPTION OF AN ILLUSTRATIVE EMBODIMENT

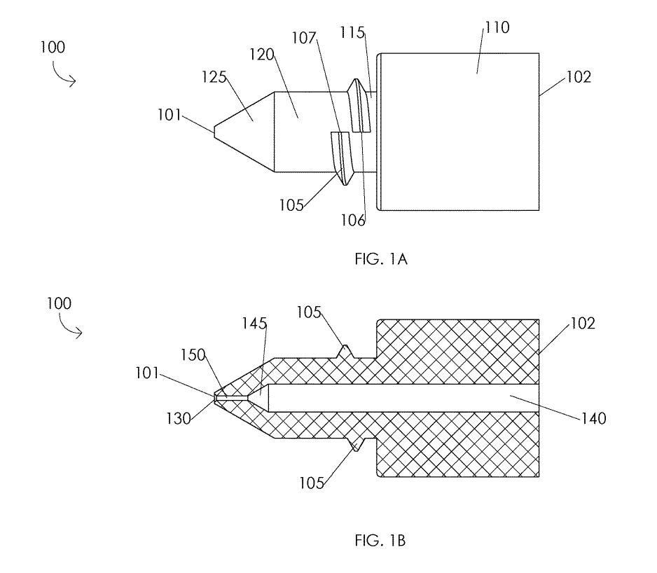

FIGS. 1A and 1B are schematic diagrams illustrating a transitory connector 100. FIG. 1A illustrates a side view of a transitory connector 100. FIG. 1B illustrates a cross-sectional view in a sagittal plane of a transitory connector 100. In one or more embodiments, transitory connector 100 may comprise a transitory connector distal end 101 and a transitory connector proximal end 102. Illustratively, transitory connector 100 may comprise a raised helix 105 having a raised helix posterior end 106 and a raised helix anterior end 107. In one or more embodiments, transitory connector 100 may comprise a proximal base 110, a distal base 115, a nosecone base 120, and a nosecone 125. Illustratively, distal base 115 may be disposed between proximal base 110 and nosecone base 120. In one or more embodiments, nosecone base 120 may be disposed between distal base 115 and nosecone 125. Illustratively, transitory connector 100 may comprise an optic fiber fixation mechanism housing 130. In one or more embodiments, transitory connector 100 may comprise an inner bore 140. Illustratively, transitory connector 100 may comprise an inner bore distal taper 145. In one or more embodiments, transitory connector 100 may comprise an optic fiber housing 150. Illustratively, inner bore distal taper 145 may be disposed between optic fiber housing 150 and inner bore 140. In one or more embodiments, raised helix 105 may be disposed between distal base 115 and nosecone base 120. Illustratively, raised helix 105 may comprise a screw threading, e.g., raised helix 105 may comprise an external thread.

In one or more embodiments, transitory connector 100 may be manufactured from a material configured to deform if transitory connector 100 is sterilized in a medical autoclave, e.g., transitory connector 100 may be manufactured from a material configured to permanently deform if transitory connector 100 is sterilized in a medical autoclave. Illustratively, transitory connector 100 may be manufactured from a material having a melting point below a temperature parameter for a steam sterilization cycle, e.g., transitory connector 100 may be manufactured from a material having a melting point below a temperature parameter for a gravity-displacement steam sterilization cycle, a dynamic-air-removal steam sterilization cycle, etc. In one or more embodiments, transitory connector 100 may be manufactured from a material having a melting point below 140.0 degrees Fahrenheit. Illustratively, transitory connector 100 may be manufactured from a material having a melting point in a range of 158.0 to 212.0 degrees Fahrenheit, e.g., transitory connector 100 may be manufactured from a material having a melting point of 160.0 degrees Fahrenheit. In one or more embodiments, transitory connector 100 may be manufactured from a material having a melting point of less than 158.0 degrees Fahrenheit or greater than 212.0 degrees Fahrenheit. In one or more embodiments, transitory connector 100 may be manufactured from a material having a melting point below 250.0 degrees Fahrenheit. Illustratively, transitory connector 100 may be manufactured from a material having a melting point below 270.0 degrees Fahrenheit. In one or more embodiments, transitory connector 100 may be manufactured from a material having a melting point below 275.0 degrees Fahrenheit.

Illustratively, transitory connector 100 may be manufactured from a material configured to temporarily deform if transitory connector 100 is sterilized in a medical autoclave, e.g., transitory connector 100 may be manufactured from a material configured to absorb water in a medical autoclave. In one or more embodiments, an absorption of water may be configured to deform transitory connector 100, e.g., an absorption of water may be configured to cause transitory connector 100 to expand. Illustratively, transitory connector 100 may be manufactured from a porous material configured to facilitate a deformation of transitory connector 100 if transitory connector 100 is sterilized in a medical autoclave. In one or more embodiments, transitory connector 100 may be manufactured with one or more cavities configured to facilitate a deformation of transitory connector 100 if transitory connector 100 is sterilized in a medical autoclave. Illustratively, transitory connector 100 may be manufactured from any suitable material, e.g., polymers, metals, metal alloys, etc., or from any combination of suitable materials. In one or more embodiments, transitory connector 100 may be manufactured by a 3D printing process. For example, transitory connector 100 may be manufactured by selective laser sintering, selective heat sintering, selective laser melting, electron-beam melting, direct metal laser sintering, electron beam freeform fabrication, etc. Illustratively, transitory connector 100 may be manufactured by injection molding. In one or more embodiments, transitory connector 100 may be manufactured by additive manufacturing.

In one or more embodiments, transitory connector 100 may be manufactured from poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)-dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc. Illustratively, transitory connector 100 may be manufactured from any substituted polymers of poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc.

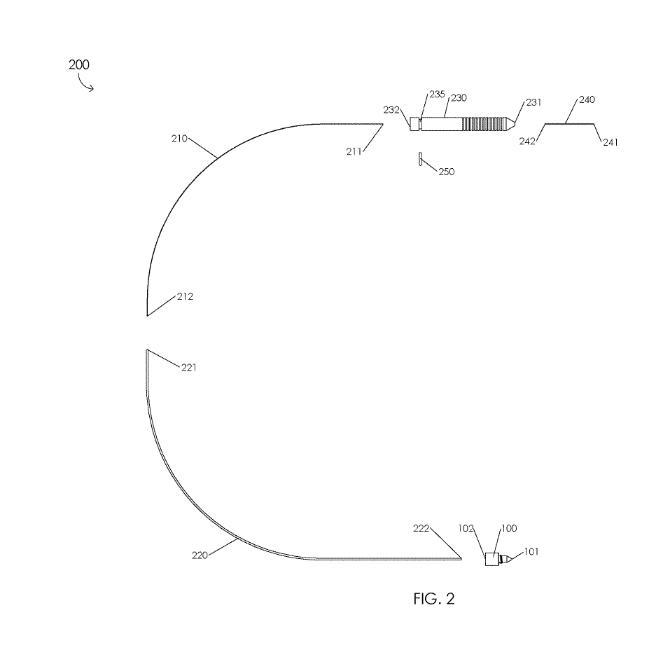

FIG. 2 is a schematic diagram illustrating an exploded view of a single-use laser probe assembly 200. Illustratively, a single-use laser probe assembly 200 may comprise a transitory connector 100, an optic fiber 210, a jacketing 220, a laser probe handle 230, a hypodermic tube 240, and an identification ring 250. In one or more embodiments, optic fiber 210 may comprise an optic fiber distal end 211 and an optic fiber proximal end 212. Illustratively, jacketing 220 may comprise a jacketing distal end 221 and a jacketing proximal end 222. In one or more embodiments, laser probe handle 230 may comprise a laser probe handle distal end 231 and a laser probe handle proximal end 232. Illustratively, laser probe handle 230 may comprise an identification ring channel 235. In one or more embodiments, hypodermic tube 240 may comprise a hypodermic tube distal end 241 and a hypodermic tube proximal end 242.

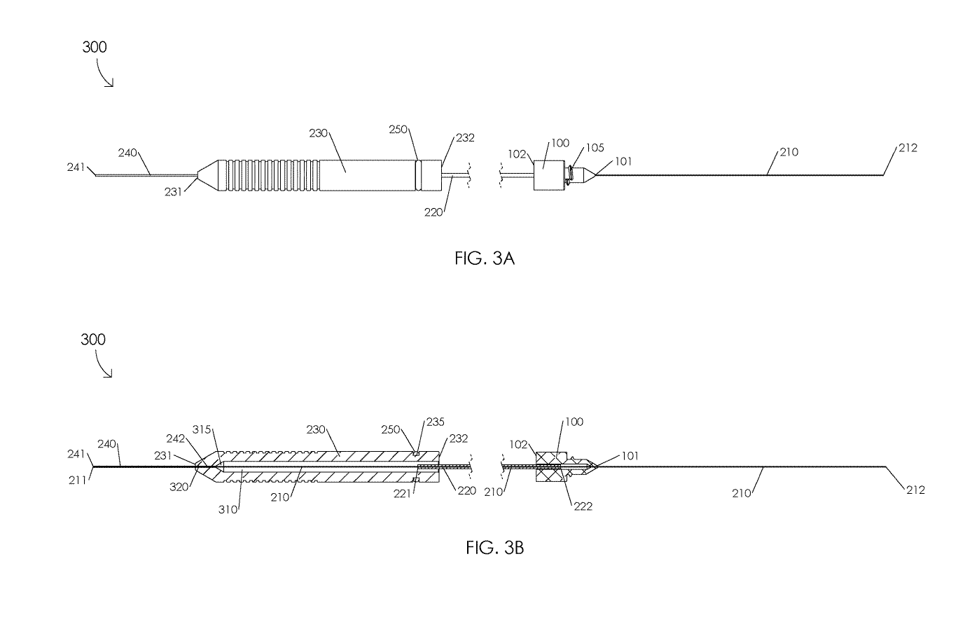

FIGS. 3A and 3B are schematic diagrams illustrating an assembled single-use laser probe 300. FIG. 3A illustrates a side view of an assembled single-use laser probe 300. FIG. 3B illustrates a cross-sectional view in a sagittal plane of an assembled single-use laser probe 300. Illustratively, optic fiber 210 may be disposed within jacketing 220 wherein optic fiber distal end 211 extends out from jacketing distal end 221 and wherein optic fiber proximal end 212 extends out from jacketing proximal end 222. In one or more embodiments, optic fiber 210 may be disposed in transitory connector 100, e.g., optic fiber 210 may be disposed in transitory connector 100 wherein optic fiber proximal end 212 extends out from transitory connector distal end 101. Illustratively, optic fiber 210 may be disposed in inner bore 140, inner bore distal taper 145, optic fiber housing 150, and optic fiber fixation mechanism housing 130. In one or more embodiments, optic fiber 210 may be fixed in transitory connector 100, e.g., optic fiber 210 may be fixed in transitory connector 100 by an adhesive, an epoxy, a crimp, a weld, an interference fit, a tie, etc. Illustratively, optic fiber 210 may be fixed in optic fiber housing 150, e.g., optic fiber 210 may be fixed in optic fiber housing 150 by an adhesive, an epoxy, a crimp, a weld, an interference fit, a tie, etc. In one or more embodiments, an adhesive may be disposed in optic fiber fixation mechanism housing 130 to fix optic fiber 210 in transitory connector 100, e.g., a UV curable adhesive may be disposed in optic fiber fixation mechanism housing 130 and cured to fix optic fiber 210 in transitory connector 100. Illustratively, a portion of jacketing 220 may be disposed in a portion of transitory connector 100, e.g., jacketing proximal end 222 may be disposed in inner bore 140. In one or more embodiments, a portion of jacketing 220 may be fixed in a portion of transitory connector 100, e.g., a portion of jacketing 220 may be fixed in a portion of transitory connector 100 by an adhesive, an epoxy, a crimp, a weld, an interference fit, a tie, etc.

Illustratively, laser probe handle 230 may comprise a handle inner bore 310, a handle inner bore distal taper 315, and a hypodermic tube housing 320. In one or more embodiments, identification ring 250 may be disposed in identification ring channel 235. Illustratively, identification ring 250 may be fixed in identification ring channel 235, e.g., identification ring 250 may be fixed in identification ring channel 235 by an adhesive, an epoxy, a crimp, a weld, an interference fit, a tie, etc. In one or more embodiments, a portion of hypodermic tube 240 may be disposed in a portion of laser probe handle 230, e.g., hypodermic tube proximal end 242 may be disposed in hypodermic tube housing 320. Illustratively, a portion of hypodermic tube 240 may be disposed in a portion of laser probe handle 230 wherein hypodermic tube distal end 241 extends out from laser probe handle distal end 231. In one or more embodiments, a portion of hypodermic tube 240 may be fixed in a portion of laser probe handle 230, e.g., a portion of hypodermic tube 240 may be fixed in a portion of laser probe handle 230 by an adhesive, an epoxy, a crimp, a weld, an interference fit, a tie, etc. Illustratively, a portion of jacketing 220 may be disposed in a portion of laser probe handle 230, e.g., jacketing distal end 221 may be disposed in handle inner bore 310. In one or more embodiments, a portion of jacketing 220 may be fixed in a portion of laser probe handle 230, e.g., a portion of jacketing 220 may be fixed in a portion of laser probe handle 230 by an adhesive, an epoxy, a crimp, a weld, an interference fit, a tie, etc. Illustratively, optic fiber 210 may be disposed in laser probe handle 230. In one or more embodiments, optic fiber 210 may be disposed in handle inner bore 310, handle inner bore distal taper 315, hypodermic tube housing 320, and hypodermic tube 240. Illustratively, optic fiber 210 may be disposed in hypodermic tube 240 wherein optic fiber distal end 211 is adjacent to hypodermic tube distal end 241, e.g., optic fiber 210 may be disposed in hypodermic tube 240 wherein optic fiber distal end 211 is coplanar with hypodermic tube distal end 241. In one or more embodiments, optic fiber 210 may be fixed in hypodermic tube 240, e.g., optic fiber 210 may be fixed in hypodermic tube 240 by an adhesive, an epoxy, a crimp, a weld, an interference fit, a tie, etc.

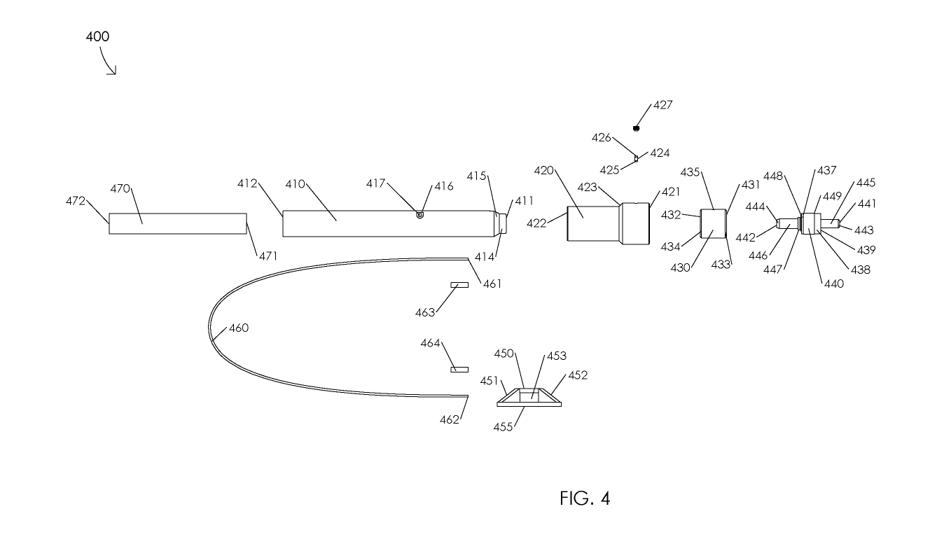

FIG. 4 is a schematic diagram illustrating an exploded view of an optic fiber fixture assembly 400. Illustratively, an optic fiber fixture assembly 400 may comprise a fixture base 410, a machine connector housing 420, an electrical element 424, a fixation mechanism 427, an extender 430, a machine connector 440, a machine coupler 450, a lanyard cable 460, a distal fastener 463, a proximal fastener 464, and a fixture facilitating sleeve 470. In one or more embodiments, fixture base 410 may comprise a fixture base distal end 411 and a fixture base proximal end 412. Illustratively, fixture base 410 may comprise an extender interface 414, a fixture base distal taper 415, an indentation 416, and a lanyard cable guide 417. In one or more embodiments, lanyard cable guide 417 may be disposed in indentation 416. Illustratively, lanyard cable guide 417 may be disposed dorsally in fixture base 410. In one or more embodiments, machine connector housing 420 may comprise a machine connector housing distal end 421 and a machine connector housing proximal end 422. Illustratively, machine connector housing 420 may comprise a machine connector housing taper 423. In one or more embodiments, extender 430 may comprise an extender distal end 431 and an extender proximal end 432. Illustratively, extender 430 may comprise an extender distal taper 433 and an extender proximal taper 434. In one or more embodiments, extender 430 may comprise an electrical element housing 435. Illustratively, electrical element housing 435 may be configured to house electrical element 424. In one or more embodiments, electrical element 424 may comprise an electrical element inferior end 425 and an electrical element superior end 426.

In one or more embodiments, machine connector 440 may comprise a machine connector distal end 441 and a machine connector proximal end 442. Illustratively, machine connector 440 may comprise a machine connector distal taper 443, a machine connector proximal taper 444, a distal ferrule 445, and a proximal ferrule 446. In one or more embodiments, machine connector 440 may comprise a machine connector base 438. Illustratively, machine connector base 438 may comprise a machine connector base distal end 439 and a machine connector base proximal end 449. In one or more embodiments, machine connector 440 may comprise a retaining ring distal interface 437, a retaining ring proximal interface 447, and a retaining ring 448. Illustratively, retaining ring 448 may be disposed between retaining ring distal interface 437 and retaining ring proximal interface 447. In one or more embodiments, lanyard cable 460 may comprise a lanyard cable distal end 461 and a lanyard cable proximal end 462. Illustratively, machine coupler 450 may comprise a machine coupler inferior end 451 and a machine coupler superior end 452. In one or more embodiments, machine coupler 450 may comprise a machine coupler aperture 453. Illustratively, machine coupler 450 may comprise a machine interface 455.

In one or more embodiments, fixture facilitating sleeve 470 may comprise a fixture facilitating sleeve distal end 471 and a fixture facilitating sleeve proximal end 472. Illustratively, fixture facilitating sleeve 470 may be manufactured from a material configured to minimize a coefficient of friction between a portion of optic fiber 210 and a portion of fixture facilitating sleeve 470, e.g., fixture facilitating sleeve 470 may be manufactured from a self-lubricating thermoplastic material. In one or more embodiments, fixture facilitating sleeve 470 may be manufactured from a material wherein a coefficient of friction between a portion of optic fiber 210 and a portion of fixture facilitating sleeve 470 is in a range of 0.011 to 0.36, e.g., fixture facilitating sleeve 470 may be manufactured from a material wherein a coefficient of friction between a portion of optic fiber 210 and a portion of fixture facilitating sleeve 470 is 0.0311. Illustratively, fixture facilitating sleeve 470 may be manufactured from a material wherein a coefficient of friction between a portion of optic fiber 210 and a portion of fixture facilitating sleeve 470 is less than 0.011 or greater than 0.36. In one or more embodiments, fixture facilitating sleeve 470 may be manufactured from a fluorocarbon material, e.g., fixture facilitating sleeve 470 may be manufactured from a polytetrafluoroethylene material. Illustratively, fixture facilitating sleeve 470 may be manufactured from an acetal-based polytetrafluoroethylene material, e.g., fixture facilitating sleeve 470 may be manufactured from a turcite material. In one or more embodiments, fixture facilitating sleeve 470 may be manufactured from a material having a density in a range of 0.024 to 0.073 pounds per cubic inch, e.g., fixture facilitating sleeve 470 may be manufactured from a material having a density of 0.053 pounds per cubic inch. Illustratively, fixture facilitating sleeve 470 may be manufactured from a material having a density of less than 0.024 pounds per cubic inch or greater than 0.073 pounds per cubic inch. In one or more embodiments, fixture facilitating sleeve 470 may be manufactured from a material having a hardness in a range of 50 Shore D to 75 Shore D, e.g., fixture facilitating sleeve 470 may be manufactured from a material having a hardness of 61 Shore D. Illustratively, fixture facilitating sleeve 470 may be manufactured from a material having a hardness of less than 50 Shore D or greater than 75 Shore D. In one or more embodiments, optic fiber 210 may be manufactured from a material having a first hardness and fixture facilitating sleeve 470 may be manufactured from a material having a second hardness. Illustratively, the first hardness may be greater than the second hardness. In one or more embodiments, optic fiber 210 may be manufactured from a material having a first hardness, fixture facilitating sleeve 470 may be manufactured from a material having a second hardness, and transitory connector 100 may be manufactured from a material having a third hardness. Illustratively, the first hardness may be greater than the second hardness and the second hardness may be greater than the third hardness.

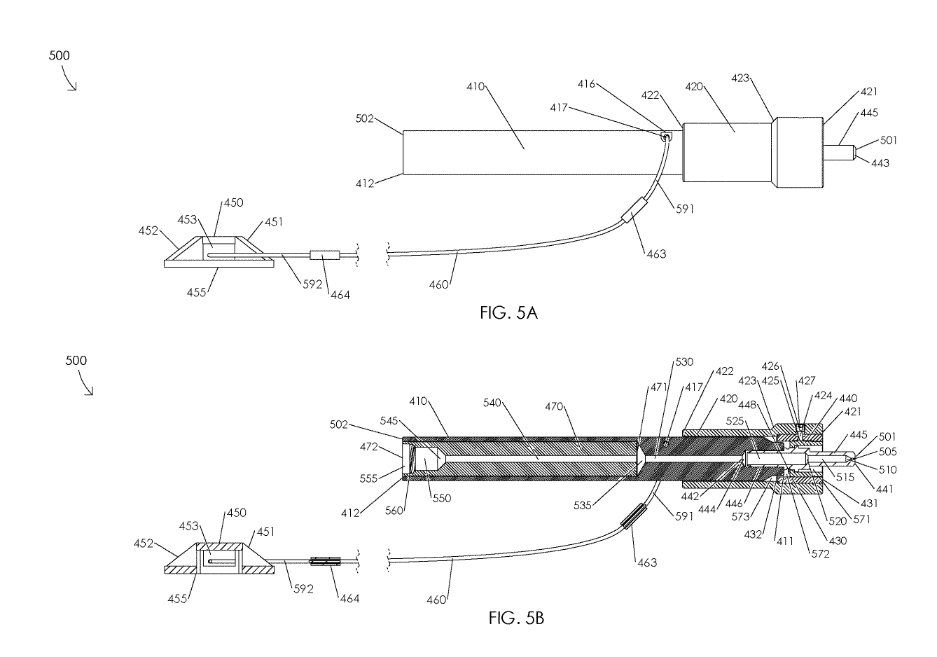

FIGS. 5A and 5B are schematic diagrams illustrating an assembled optic fiber fixture 500. FIG. 5A illustrates a side view of an assembled optic fiber fixture 500. FIG. 5B illustrates a cross-sectional view in a sagittal plane of an assembled optic fiber fixture 500. Illustratively, an assembled optic fiber fixture 500 may comprise an assembled optic fiber fixture distal end 501 and an assembled optic fiber fixture proximal end 502. In one or more embodiments, assembled optic fiber fixture 500 may comprise an optic fiber proximal end guide 505, an inner lumen distal taper 510, a machine connector distal inner lumen 515, an inner lumen proximal taper 520, a machine connector proximal inner lumen 525, a fixture base inner bore 530, a fixture facilitating sleeve housing 535, a fixture facilitating sleeve inner bore 540, a transitory connector housing distal taper 545, a transitory connector housing 550, a transitory connector housing proximal chamber 555, and a transitory connector housing threading 560.

Illustratively, fixture facilitating sleeve 470 may be disposed in a portion of fixture base 410, e.g., fixture facilitating sleeve 470 may be disposed in fixture facilitating sleeve housing 535. In one or more embodiments, fixture facilitating sleeve 470 may be disposed in a portion of fixture base 410 wherein fixture facilitating sleeve proximal end 472 is adjacent to fixture base proximal end 412, e.g., fixture facilitating sleeve 470 may be disposed in a portion of fixture base 410 wherein fixture facilitating sleeve proximal end 472 abuts fixture base proximal end 412. Illustratively, fixture facilitating sleeve 470 may be disposed in a portion of fixture base 410 wherein fixture facilitating sleeve inner bore 540 is aligned with fixture base inner bore 530, e.g., fixture facilitating sleeve 470 may be disposed in a portion of fixture base 410 wherein fixture facilitating sleeve inner bore 540 is collinear with fixture base inner bore 530. In one or more embodiments, fixture facilitating sleeve 470 may be disposed in a portion of fixture base 410 wherein fixture facilitating sleeve 470 is fixed in the portion of fixture base 410, e.g., fixture facilitating sleeve 470 may be fixed in a portion of fixture base 410 by an adhesive, an epoxy, a friction fit, a crimp, a tie, a weld, etc. In one or more embodiments, fixture facilitating sleeve 470 may be fixed in fixture facilitating sleeve housing 535, e.g., fixture facilitating sleeve 470 may be fixed in fixture facilitating sleeve housing 535 by an adhesive, an epoxy, a friction fit, a crimp, a tie, a weld, etc.

In one or more embodiments, lanyard cable 460 may comprise a distal loop 591 and a proximal loop 592. Illustratively, distal fastener 463 may be disposed over a portion of lanyard cable 460 wherein lanyard cable distal end 461 extends a distance from distal fastener 463. In one or more embodiments, lanyard cable distal end 461 may be threaded through lanyard cable guide 417 and into a portion of distal fastener 463 to form distal loop 591. Illustratively, distal fastener 463 may be configured to fix lanyard cable distal end 461 within distal fastener 463, e.g., distal fastener 463 may be configured to fix lanyard cable distal end 461 within distal fastener 463 by an adhesive, a crimp, a weld, a friction fit, etc. In one or more embodiments, proximal fastener 464 may be disposed over a portion of lanyard cable 460 wherein lanyard cable proximal end 462 extends a distance from proximal fastener 464. Illustratively, lanyard cable proximal end 462 may be threaded through machine coupler aperture 453 and into a portion of proximal fastener 464 to form proximal loop 592. In one or more embodiments, proximal fastener 464 may be configured to fix lanyard cable proximal end 462 within proximal fastener 464, e.g., proximal fastener 464 may be configured to fix lanyard cable proximal end 462 within proximal fastener 464 by an adhesive, an epoxy, a crimp, a weld, a friction fit, etc.

Illustratively, machine connector 440 may comprise a machine connector inner chamber 571. In one or more embodiments, distal ferrule 445 may extend a distance out from machine connector inner chamber 571. Illustratively, extender 430 may comprise an extender inner chamber 572. In one or more embodiments, machine connector 440 may be disposed in extender inner chamber 572. Illustratively, machine connector 440 may be disposed in extender 430, e.g., machine connector 440 may be disposed in extender 430 wherein machine connector distal end 441 may extend a distance from extender distal end 431 and wherein machine connector proximal end 442 may extend a distance from extender proximal end 432. In one or more embodiments, machine connector 440 may be fixed in extender 430, e.g., machine connector 440 may be fixed in extender 430 by an adhesive, a crimp, a weld, a friction fit, etc. Illustratively, machine connector housing 420 may comprise a machine connector housing inner chamber 573. In one or more embodiments, extender 430 may be disposed in machine connector housing inner chamber 573. Illustratively, extender 430 may be disposed in machine connector housing 420, e.g., extender 430 may be disposed in machine connector housing 420 wherein extender distal end 431 is disposed between machine connector housing distal end 421 and machine connector housing proximal end 422 and wherein extender proximal end 432 is disposed between machine connector housing distal end 421 and machine connector housing proximal end 422. In one or more embodiments, extender 430 may be disposed in machine connector housing 420 wherein machine connector distal end 441 extends a distance from machine connector housing distal end 421, e.g., extender may be disposed in machine connector housing 420 wherein machine connector proximal end 442 may be disposed between machine connector housing distal end 421 and machine connector housing proximal end 422. Illustratively, extender 430 may be fixed in machine connector housing 420, e.g., extender 430 may be fixed in machine connector housing 420 by an adhesive, an epoxy, a crimp, a weld, a friction fit, etc.

In one or more embodiments, fixture base 410 may be disposed in machine connector housing 420, e.g., fixture base 410 may be disposed in machine connector housing 420 wherein fixture base proximal end 412 extends a distance from machine connector housing proximal end 422 and wherein fixture base distal end 411 is disposed between machine connector housing distal end 421 and machine connector housing proximal end 422. Illustratively, fixture base 410 may be fixed in machine connector housing 420, e.g., fixture base 410 may be fixed in machine connector housing 420 by an adhesive, a crimp, a weld, a friction fit, etc. In one or more embodiments, fixture base 410 may be disposed in extender 430, e.g., fixture base 410 may be disposed in extender 430 wherein fixture base proximal end 412 extends a distance from extender proximal end 432 and wherein fixture base distal end 411 is disposed between extender distal end 431 and extender proximal end 432. Illustratively, fixture base 410 may be fixed in extender 430, e.g., fixture base 410 may be fixed in extender 430 by an adhesive, a crimp, a weld, a friction fit, etc. In one or more embodiments, machine connector 440 may be disposed in fixture base 410, e.g., machine connector 440 may be disposed in fixture base 410 wherein machine connector distal end 441 extends a distance from fixture base distal end 411 and wherein machine connector proximal end 442 is disposed between fixture base distal end 411 and fixture base proximal end 412. Illustratively, machine connector 440 may be fixed in fixture base 410, e.g., machine connector 440 may be fixed in fixture base 410 by an adhesive, an epoxy, a crimp, a weld, a friction fit, etc.

In one or more embodiments, electrical element 424 may be disposed in machine connector housing 420 and extender 430, e.g., electrical element may be disposed in electrical element housing 435. Illustratively, electrical element 424 may be fixed in electrical element housing 435, e.g., electrical element 424 may be fixed in electrical element housing 435 by an adhesive, an epoxy, a crimp, a weld, a friction fit, etc. In one or more embodiments, electrical element 424 may be disposed in machine connector housing 420 and extender 430 wherein electrical element inferior end 425 may be in contact with machine connector 440, e.g., electrical element 424 may be disposed in electrical element housing 435 wherein electrical element inferior end 425 may be in contact with machine connector 440. Illustratively, electrical element 424 may be electrically connected to machine connector 440. In one or more embodiments, electrical element 424 may be configured to convey data to a machine, e.g., electrical element 424 may be configured to convey data to a laser machine. Illustratively, electrical element 424 may comprise a resistor, e.g., electrical element 424 may comprise a cylindrical resistor. In one or more embodiments, electrical element 424 may comprise a radio frequency identification chip.

Illustratively, fixation mechanism 427 may be disposed in machine connector housing 420, e.g., fixation mechanism 427 may be disposed in machine connector housing 420 wherein a portion of fixation mechanism 427 contacts a portion of electrical element 424. In one or more embodiments, fixation mechanism 427 may be fixed in machine connector housing 420, e.g., fixation mechanism 427 may be fixed in machine connector housing 420 by an adhesive, an epoxy, a crimp, a weld, a friction fit, etc. Illustratively, fixation mechanism 427 may be configured to fix electrical element 424 in electrical element housing 435, e.g., fixation mechanism 427 may comprise a setscrew configured to fix electrical element 424 in electrical element housing 435. In one or more embodiments, fixation mechanism 427 may be electrically conductive. Illustratively, fixation mechanism 427 may be disposed in machine connector housing 420 wherein fixation mechanism 427 contacts electrical element 424 and electrical element 424 contacts machine connector 440, e.g., fixation mechanism 427 may be disposed in machine connector housing 420 wherein fixation mechanism 427 contacts electrical element superior end 426 and electrical element inferior end 425 contacts machine connector 440. In one or more embodiments, fixation mechanism 427 may be disposed in machine connector housing 420 wherein fixation mechanism 427 is electrically connected to electrical element 424 and electrical element 424 is electrically connected to machine connector 440.

Illustratively, machine coupler 450 may be configured to attach assembled optic fiber fixture 500 to a laser machine, e.g., machine interface 455 may be configured to attach assembled optic fiber fixture 500 to a laser machine. In one or more embodiments, machine interface 455 may comprise a magnet configured to attach assembled optic fiber fixture 500 to a laser machine. Illustratively, machine interface 455 may comprise an adhesive configured to attach assembled optic fiber fixture 500 to a laser machine. In one or more embodiments, assembled optic fiber fixture 500 may be reusable, e.g., assembled optic fiber fixture 500 may be sold non-sterile and not intended to be sterilized by a user in a medical autoclave. Illustratively, a user may clean assembled optic fiber fixture 500 by flushing assembled optic fiber fixture 500 with a syringe of isopropyl alcohol. In one or more embodiments, flushing assembled optic fiber fixture 500 with a syringe of isopropyl alcohol before each use of assembled optic fiber fixture 500 may be configured to remove any particulate matter that may have accumulated in assembled optic fiber fixture 500 since a previous use of assembled optic fiber fixture 500. Illustratively, optic fiber fixture 500 may comprise an end cap configured to fit over optic fiber fixture proximal end 502, e.g., optic fiber fixture 500 may comprise an end cap configured to fit over optic fiber fixture proximal end 502 to prevent particulate matter from accumulating in optic fiber fixture 500 when optic fiber fixture 500 is not being used by a user.

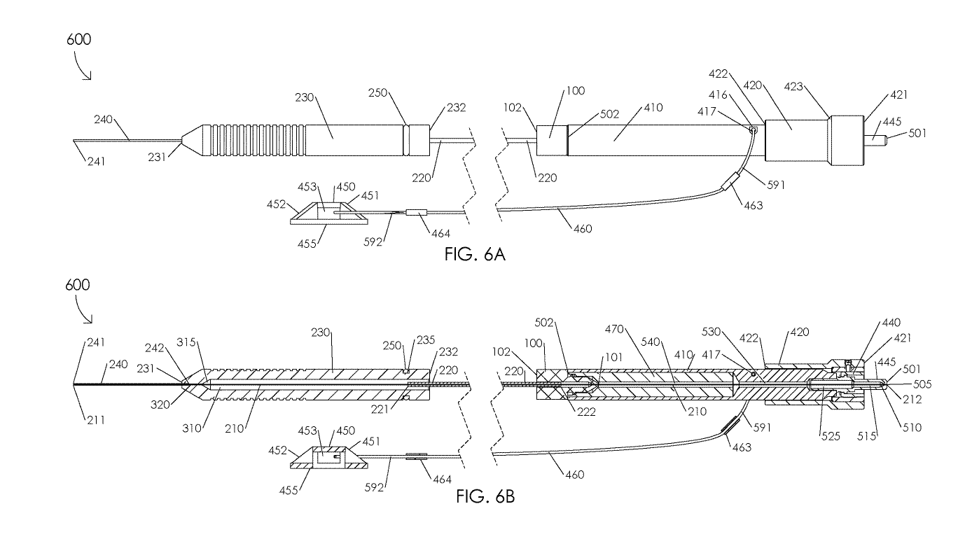

FIGS. 6A and 6B are schematic diagrams illustrating a single-use laser probe with optic fiber fixture 600. FIG. 6A illustrates a side view of a single-use laser probe with optic fiber fixture 600. FIG. 6B illustrates a cross-sectional view in a sagittal plane of a single-use laser probe with optic fiber fixture 600. Illustratively, a single-use laser probe with optic fiber fixture 600 may comprise an assembled single-use laser probe 300 and an assembled optic fiber fixture 500. In one or more embodiments, a single-use laser probe with optic fiber fixture 600 may comprise a transitory connector 100 disposed in a portion of assembled optic fiber fixture 500 wherein optic fiber proximal end 212 is adjacent to assembled optic fiber fixture distal end 501. Illustratively, a single-use laser probe with optic fiber fixture 600 may comprise a transitory connector 100 disposed in a portion of assembled optic fiber fixture 500 wherein optic fiber proximal end 212 abuts assembled optic fiber fixture distal end 501. In one or more embodiments, a single-use laser probe with optic fiber fixture 600 may comprise a transitory connector 100 disposed in a portion of assembled optic fiber fixture 500 wherein optic fiber proximal end 212 is coplanar with assembled optic fiber fixture distal end 501. Illustratively, inserting a portion of optic fiber 210 into a portion of assembled optic fiber fixture 500 may be configured to ingress optic fiber proximal end 212 into transitory connector housing 550. In one or more embodiments, inserting a portion of optic fiber 210 into a portion of assembled optic fiber fixture 500 may be configured to ingress optic fiber proximal end 212 into transitory connector housing distal taper 545. Illustratively, transitory connector housing distal taper 545 may be configured to guide an ingress of optic fiber 210 into fixture facilitating sleeve inner bore 540. In one or more embodiments, inserting a portion of optic fiber 210 into a portion of assembled optic fiber fixture 500 may be configured to ingress optic fiber proximal end 212 into fixture facilitating sleeve inner bore 540. Illustratively, inserting a portion of optic fiber 210 into a portion of assembled optic fiber fixture 500 may be configured to ingress optic fiber proximal end 212 into fixture facilitating sleeve housing 535. In one or more embodiments, fixture facilitating sleeve housing 535 may be configured to guide an ingress of optic fiber 210 into fixture base inner bore 530. Illustratively, inserting a portion of optic fiber 210 into a portion of assembled optic fiber fixture 500 may be configured to ingress optic fiber proximal end 212 into fixture base inner bore 530. In one or more embodiments, inserting a portion of optic fiber 210 into a portion of assembled optic fiber fixture 500 may be configured to ingress optic fiber proximal end 212 into machine connector proximal taper 444. Illustratively, machine connector proximal taper 444 may be configured to guide an ingress of optic fiber 210 into machine connector proximal inner lumen 525. In one or more embodiments, inserting a portion of optic fiber 210 into a portion of assembled optic fiber fixture 500 may be configured to ingress optic fiber proximal end 212 into machine connector proximal inner lumen 525. Illustratively, inserting a portion of optic fiber 210 into a portion of assembled optic fiber fixture 500 may be configured to ingress optic fiber proximal end 212 into inner lumen proximal taper 520. In one or more embodiments, inner lumen proximal taper 520 may be configured to guide an ingress of optic fiber into machine connector distal inner lumen 515. Illustratively, inserting a portion of optic fiber 210 into a portion of assembled optic fiber fixture 500 may be configured to ingress optic fiber proximal end 212 into machine connector distal inner lumen 515. In one or more embodiments, inserting a portion of optic fiber 210 into a portion of assembled optic fiber fixture 500 may be configured to ingress optic fiber proximal end 212 into inner lumen distal taper 510. Illustratively, inner lumen distal taper 510 may be configured to guide an ingress of optic fiber 210 into optic fiber proximal end guide 505. In one or more embodiments, inserting a portion of optic fiber 210 into a portion of assembled optic fiber fixture 500 may be configured to ingress optic fiber proximal end 212 into optic fiber proximal end guide 505.

Illustratively, inserting transitory connector 100 into a portion of assembled optic fiber fixture 500 may be configured to ingress optic fiber 210 into optic fiber proximal end guide 505, e.g., inserting transitory connector 100 into a portion of assembled optic fiber fixture 500 may be configured to ingress optic fiber 210 into optic fiber proximal end guide 505 wherein optic fiber proximal end 212 is coplanar with assembled optic fiber fixture distal end 501. In one or more embodiments, inserting transitory connector distal end 101 into transitory connector housing distal taper 545 may be configured to ingress optic fiber 210 into optic fiber proximal end guide 505, e.g., inserting transitory connector distal end 101 into transitory connector housing distal taper 545 may be configured to ingress optic fiber 210 into optic fiber proximal end guide 505 wherein optic fiber proximal end 212 is coplanar with assembled optic fiber fixture distal end 501. Illustratively, inserting transitory connector nosecone base 120 into transitory connector housing 550 may be configured to ingress optic fiber 210 into optic fiber proximal end guide 505, e.g., inserting transitory connector nosecone base 120 into transitory connector housing 550 may be configured to ingress optic fiber 210 into optic fiber proximal end guide 505 wherein optic fiber proximal end 212 is coplanar with assembled optic fiber fixture distal end 501. In one or more embodiments, inserting raised helix 105 into transitory connector housing threading 560 may be configured to ingress optic fiber 210 into optic fiber proximal end guide 505, e.g., inserting raised helix 105 into transitory connector housing threading 560 may be configured to ingress optic fiber 210 into optic fiber proximal end guide 505 wherein optic fiber proximal end 212 is coplanar with assembled optic fiber fixture distal end 501. Illustratively, rotating raised helix 105 in transitory connector housing threading 560 may be configured to ingress optic fiber 210 into optic fiber proximal end guide 505, e.g., rotating raised helix 105 in transitory connector housing threading 560 may be configured to ingress optic fiber 210 into optic fiber proximal end guide 505 wherein optic fiber proximal end 212 is coplanar with assembled optic fiber fixture distal end 501. In one or more embodiments, rotating raised helix 105 in transitory connector housing threading 560 may be configured to fix transitory connector 100 in assembled optic fiber fixture 500, e.g., rotating raised helix 105 in transitory connector housing threading 560 may be configured to fix raised helix 105 in first transitory connector housing threading 560. For example, raised helix 105 may comprise a threaded fastener.

Illustratively, a user may perform a photocoagulation procedure with a single-use laser probe with optic fiber fixture 600. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site.

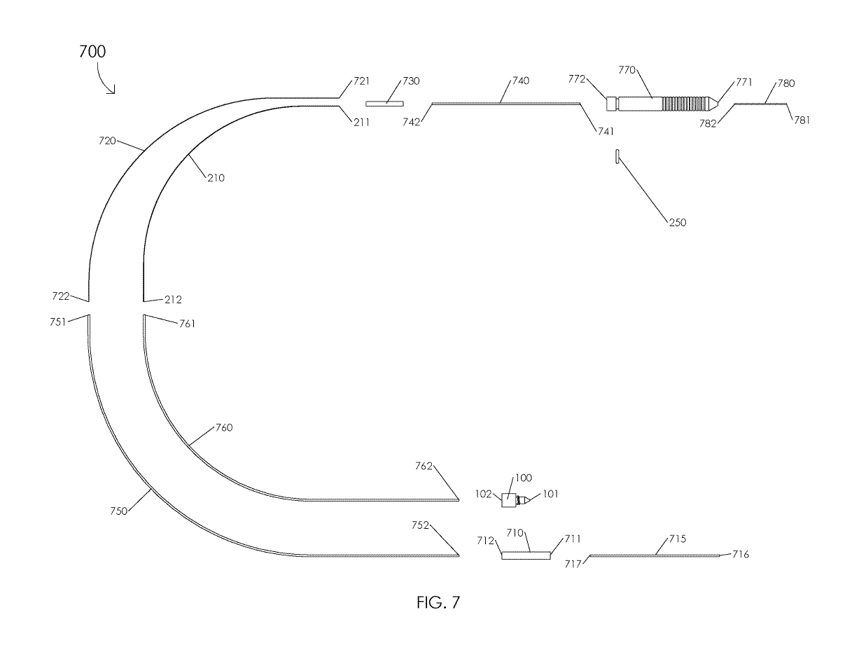

FIG. 7 is a schematic diagram illustrating an exploded view of a single-use illuminated laser probe assembly 700. Illustratively, a single-use illuminated laser probe assembly 700 may comprise a transitory connector 100, an optic fiber 210, an identification ring 250, an illumination optic fiber fixture interface 710, an illumination optic fiber housing 715, an illumination optic fiber 720, a coupling sleeve 730, a dual fiber housing 740, an illumination jacketing 750, a jacketing 760, an illuminated laser probe handle 770, and a hypodermic tube 780. In one or more embodiments, optic fiber 210 may comprise an optic fiber distal end 211 and an optic fiber proximal end 212. Illustratively, illumination optic fiber fixture interface 710 may comprise an illumination optic fiber fixture interface distal end 711 and an illumination optic fiber fixture interface proximal end 712. In one or more embodiments, illumination optic fiber housing 715 may comprise an illumination optic fiber housing distal end 716 and an illumination optic fiber housing proximal end 717. Illustratively, illumination optic fiber 720 may comprise an illumination optic fiber distal end 721 and an illumination optic fiber proximal end 722. In one or more embodiments, dual fiber housing 740 may comprise a dual fiber housing distal end 741 and a dual fiber housing proximal end 742. Illustratively, illumination jacketing 750 may comprise an illumination jacketing distal end 751 and an illumination jacketing proximal end 752. In one or more embodiments, jacketing 760 may comprise a jacketing distal end 761 and a jacketing proximal end 762. Illustratively, illuminated laser probe handle 770 may comprise an illuminated laser probe handle distal end 771 and an illuminated laser probe handle proximal end 772. In one or more embodiments, hypodermic tube 780 may comprise a hypodermic tube distal end 781 and a hypodermic tube proximal end 782.

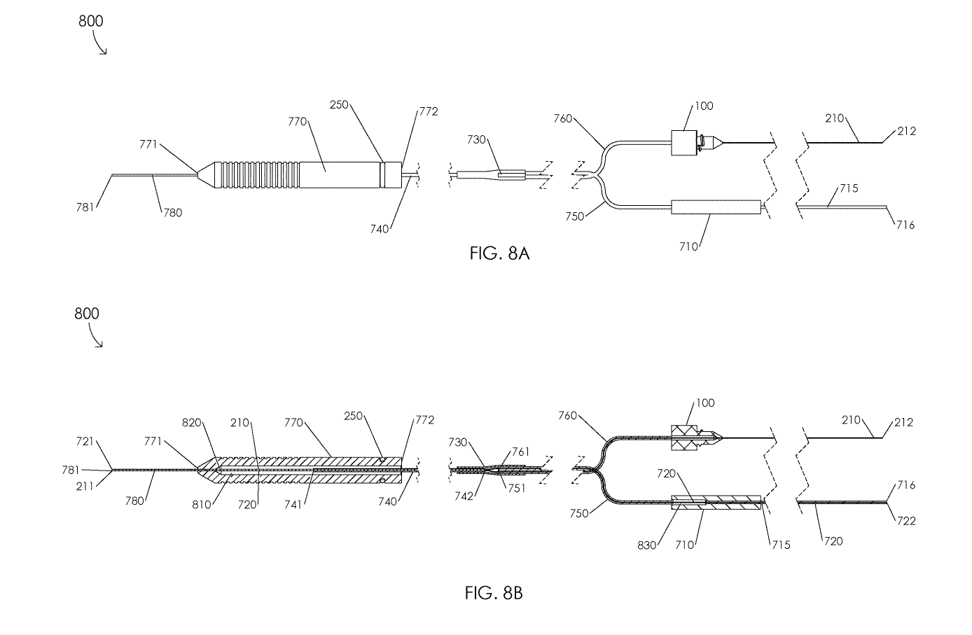

FIGS. 8A and 8B are schematic diagrams illustrating an assembled single-use illuminated laser probe 800. FIG. 8A illustrates a side view of an assembled single-use illuminated laser probe 800. FIG. 8B illustrates a cross-sectional view in a sagittal plane of an assembled single-use illuminated laser probe 800. Illustratively, optic fiber 210 may be disposed within jacketing 760 wherein optic fiber distal end 211 extends out from jacketing distal end 761 and wherein optic fiber proximal end 212 extends out from jacketing proximal end 762. In one or more embodiments, optic fiber 210 may be disposed in transitory connector 100, e.g., optic fiber 210 may be disposed in transitory connector 100 wherein optic fiber proximal end 212 extends out from transitory connector distal end 101. Illustratively, optic fiber 210 may be disposed in inner bore 140, inner bore distal taper 145, optic fiber housing 150, and optic fiber fixation mechanism housing 130. In one or more embodiments, optic fiber 210 may be fixed in transitory connector 100, e.g., optic fiber 210 may be fixed in transitory connector 100 by an adhesive, an epoxy, a crimp, a weld, an interference fit, a tie, etc. Illustratively, optic fiber 210 may be fixed in optic fiber housing 150, e.g., optic fiber 210 may be fixed in optic fiber housing 150 by an adhesive, an epoxy, a crimp, a weld, an interference fit, a tie, etc. In one or more embodiments, an adhesive may be disposed in optic fiber fixation mechanism housing 130 to fix optic fiber 210 in transitory connector 100, e.g., a UV curable adhesive may be disposed in optic fiber fixation mechanism housing 130 and cured to fix optic fiber 210 in transitory connector 100. Illustratively, a portion of jacketing 760 may be disposed in a portion of transitory connector 100, e.g., jacketing proximal end 762 may be disposed in inner bore 140. In one or more embodiments, a portion of jacketing 760 may be fixed in a portion of transitory connector 100, e.g., a portion of jacketing 760 may be fixed in a portion of transitory connector 100 by an adhesive, an epoxy, a crimp, a weld, an interference fit, a tie, etc.

Illustratively, illumination optic fiber 720 may be disposed in illumination jacketing 750. In one or more embodiments, illumination optic fiber 720 may be disposed in illumination jacketing 750 wherein illumination optic fiber distal end 721 extends out from illumination jacketing distal end 751. Illustratively, illumination optic fiber 720 may be disposed in illumination jacketing 750 wherein illumination optic fiber proximal end 722 extends out from illumination jacketing proximal end 752. In one or more embodiments, illumination optic fiber fixture interface 710 may comprise an illumination optic fiber fixture interface lumen 830. Illustratively, a portion of illumination optic fiber housing 715 may be disposed in illumination optic fiber fixture interface 710, e.g., illumination optic fiber housing proximal end 717 may be disposed in illumination optic fiber fixture interface 710. In one or more embodiments, a portion of illumination optic fiber housing 715 may be fixed in a portion of illumination optic fiber fixture interface 710, e.g., a portion of illumination optic fiber housing 715 may be fixed in a portion of illumination optic fiber fixture interface 710 by an adhesive, an epoxy, a crimp, a weld, an interference fit, a tie, etc. Illustratively, a portion of illumination jacketing 750 may be disposed in illumination optic fiber fixture interface 710, e.g., illumination jacketing proximal end 752 may be disposed in illumination optic fiber fixture interface 710. In one or more embodiments, a portion of illumination jacketing 750 may be fixed in illumination optic fiber fixture interface 710, e.g., a portion of illumination jacketing 750 may be fixed in illumination optic fiber fixture interface 710 by an adhesive, an epoxy, a crimp, a weld, an interference fit, a tie, etc.

Illustratively, illumination optic fiber 720 may be disposed in illumination optic fiber fixture interface 710 and illumination optic fiber housing 715, e.g., illumination optic fiber 720 may be disposed in illumination optic fiber fixture interface 710 and illumination optic fiber housing 715 wherein illumination optic fiber proximal end 722 may be adjacent to illumination optic fiber housing distal end 716. In one or more embodiments, illumination optic fiber 720 may be disposed in illumination optic fiber fixture interface 710 and illumination optic fiber housing 715 wherein illumination optic fiber proximal end 722 may abut illumination optic fiber housing distal end 716, e.g., illumination optic fiber 720 may be disposed in illumination optic fiber fixture interface 710 and illumination optic fiber housing 715 wherein illumination optic fiber proximal end 722 may be coplanar with illumination optic fiber housing distal end 716. Illustratively, illumination optic fiber 720 may be fixed in illumination optic fiber housing 715, e.g., illumination optic fiber 720 may be fixed in illumination optic fiber housing 715 by an adhesive, an epoxy, a crimp, a weld, an interference fit, a tie, etc.

In one or more embodiments, a portion of dual fiber housing 740 may be disposed in coupling sleeve 730, e.g., dual fiber housing proximal end 742 may be disposed in coupling sleeve 730. Illustratively, a portion of dual fiber housing 740 may be disposed in coupling sleeve 730 wherein dual fiber housing distal end 741 extends out from coupling sleeve 730. In one or more embodiments, a portion of dual fiber housing 740 may be fixed in coupling sleeve 730. Illustratively, a portion of dual fiber housing 740 may be fixed in coupling sleeve 730 by an adhesive, an epoxy, a crimp, a weld, an interference fit, a tie, etc.

In one or more embodiments, optic fiber 210 may be disposed in coupling sleeve 730 and dual fiber housing 740, e.g., optic fiber 210 may be disposed in coupling sleeve 730 and dual fiber housing 740 wherein optic fiber distal end 211 may extend out from dual fiber housing distal end 741. Illustratively, a portion of jacketing 760 may be disposed in coupling sleeve 730, e.g., jacketing distal end 761 may be disposed in coupling sleeve 730. In one or more embodiments, a portion of jacketing 760 may be fixed in coupling sleeve 730, e.g., a portion of jacketing 760 may be fixed in coupling sleeve 730 by an adhesive, an epoxy, a crimp, a weld, an interference fit, a tie, etc.

Illustratively, illumination optic fiber 720 may be disposed in coupling sleeve 730 and dual fiber housing 740, e.g., illumination optic fiber 720 may be disposed in coupling sleeve 730 and dual fiber housing 740 wherein illumination optic fiber distal end 721 may extend out from dual fiber housing distal end 741. In one or more embodiments, a portion of illumination jacketing 750 may be disposed in coupling sleeve 730, e.g., illumination jacketing distal end 751 may be disposed in coupling sleeve 730. Illustratively, a portion of illumination jacketing 750 may be fixed in coupling sleeve 730, e.g., a portion of illumination jacketing 750 may be fixed in coupling sleeve 730 by an adhesive, an epoxy, a crimp, a weld, an interference fit, a tie, etc.

In one or more embodiments, optic fiber 210 and illumination optic fiber 720 may be disposed in coupling sleeve 730 and dual fiber housing 740, e.g., optic fiber 210 and illumination optic fiber 720 may be disposed in coupling sleeve 730 and dual fiber housing 740 wherein optic fiber distal end 211 extends out from dual fiber housing distal end 741 and wherein illumination optic fiber distal end 721 extends out from dual fiber housing distal end 741. Illustratively, a portion of jacketing 760 and a portion of illumination jacketing 750 may be disposed in coupling sleeve 730, e.g., jacketing distal end 761 and illumination jacketing distal end 751 may be disposed in coupling sleeve 730. In one or more embodiments, a portion of jacketing 760 and a portion of illumination jacketing 750 may be fixed in coupling sleeve 730, e.g., a portion of jacketing 760 and a portion of illumination jacketing 750 may be fixed in coupling sleeve 730 by an adhesive, an epoxy, a crimp, a weld, an interference fit, a tie, etc.

Illustratively, illuminated laser probe handle 770 may comprise a handle inner bore 810 and a handle inner bore distal taper 820. In one or more embodiments, a portion of hypodermic tube 780 may be disposed in a portion of illuminated laser probe handle 770, e.g., hypodermic tube proximal end 782 may be disposed in a portion of illuminated laser probe handle 770. Illustratively, a portion of hypodermic tube 780 may be disposed in a portion of illuminated laser probe handle 770 wherein hypodermic tube distal end 781 extends out from illuminated laser probe handle distal end 771. In one or more embodiments, a portion of hypodermic tube 780 may be fixed in a portion of illuminated laser probe handle 770, e.g., a portion of hypodermic tube 780 may be fixed in a portion of illuminated laser probe handle 770 by an adhesive, an epoxy, a crimp, a weld, an interference fit, a tie, etc. Illustratively, a portion of dual fiber housing 740 may be disposed in a portion of illuminated laser probe handle 770, e.g., dual fiber housing distal end 741 may be disposed in handle inner bore 810. In one or more embodiments, a portion of dual fiber housing 740 may be fixed in a portion of illuminated laser probe handle 770, e.g., a portion of dual fiber housing 740 may be fixed in a portion of illuminated laser probe handle 770 by an adhesive, an epoxy, a magnet, a crimp, a weld, an interference fit, a tie, etc.

Illustratively, optic fiber 210 may be disposed in illuminated laser probe handle 770. In one or more embodiments, optic fiber 210 may be disposed in handle inner bore 810, handle inner bore distal taper 820, and hypodermic tube 780. Illustratively, optic fiber 210 may be disposed in hypodermic tube 780 wherein optic fiber distal end 211 is adjacent to hypodermic tube distal end 781, e.g., optic fiber 210 may be disposed in hypodermic tube 780 wherein optic fiber distal end 211 is coplanar with hypodermic tube distal end 781. In one or more embodiments, optic fiber 210 may be fixed in hypodermic tube 780, e.g., optic fiber 210 may be fixed in hypodermic tube 780 by an adhesive, an epoxy, a crimp, a weld, an interference fit, a tie, etc.

Illustratively, illumination optic fiber 720 may be disposed in illuminated laser probe handle 770. In one or more embodiments, illumination optic fiber 720 may be disposed in handle inner bore 810, handle inner bore distal taper 820, and hypodermic tube 780. Illustratively, illumination optic fiber 720 may be disposed in hypodermic tube 780 wherein illumination optic fiber distal end 721 is adjacent to hypodermic tube distal end 781, e.g., illumination optic fiber 720 may be disposed in hypodermic tube 780 wherein illumination optic fiber distal end 721 is coplanar with hypodermic tube distal end 781. In one or more embodiments, illumination optic fiber 720 may be fixed in hypodermic tube 780, e.g., illumination optic fiber 720 may be fixed in hypodermic tube 780 by an adhesive, an epoxy, a crimp, a weld, an interference fit, a tie, etc.

Illustratively, optic fiber 210 and illumination optic fiber 720 may be disposed in illuminated laser probe handle 770. In one or more embodiments, optic fiber 210 and illumination optic fiber 720 may be disposed in handle inner bore 810, handle inner bore distal taper 820, and hypodermic tube 780. Illustratively, optic fiber 210 and illumination optic fiber 720 may be disposed in hypodermic tube 780 wherein optic fiber distal end 211 is adjacent to hypodermic tube distal end 781 and wherein illumination optic fiber distal end 721 is adjacent to hypodermic tube distal end 781, e.g., optic fiber 210 and illumination optic fiber 720 may be disposed in hypodermic tube 780 wherein optic fiber distal end 211 is coplanar with hypodermic tube distal end 781 and wherein illumination optic fiber distal end 721 is coplanar with hypodermic tube distal end 781. In one or more embodiments, optic fiber 210 and illumination optic fiber 720 may be fixed in hypodermic tube 780, e.g., optic fiber 210 and illumination optic fiber 720 may be fixed in hypodermic tube 780 by an adhesive, an epoxy, a crimp, a weld, an interference fit, a tie, etc.

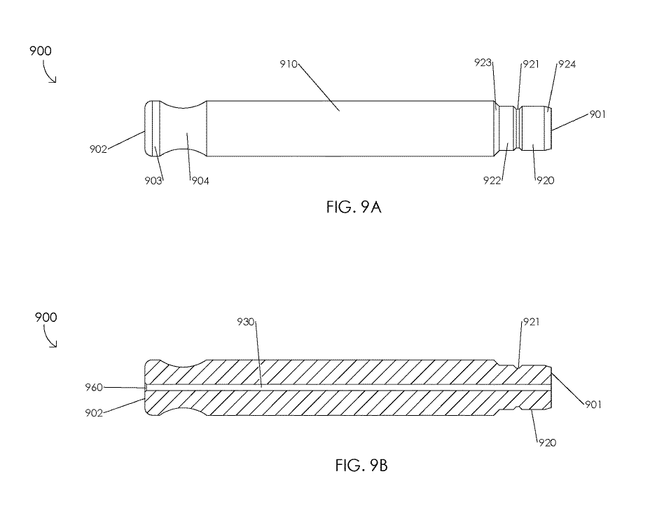

FIGS. 9A and 9B are schematic diagrams illustrating an illumination optic fiber fixture 900. FIG. 9A illustrates a side view of an illumination optic fiber fixture 900. FIG. 9B illustrates a cross-sectional view in a sagittal plane of an illumination optic fiber fixture 900. In one or more embodiments, an illumination optic fiber fixture 900 may comprise an illumination optic fiber fixture distal end 901 and an illumination optic fiber fixture proximal end 902. Illustratively, illumination optic fiber fixture 900 may comprise an illumination optic fiber fixture proximal base 903 and a concave portion 904. In one or more embodiments, illumination optic fiber fixture 900 may comprise an illumination machine connector distal interface 920, an illumination machine connector channel 921, an illumination machine connector proximal interface 922, an illumination machine connector proximal taper 923, and an illumination machine connector distal taper 924. Illustratively, illumination optic fiber fixture 900 may comprise an illumination optic fiber housing receptacle 930 and an illumination optic fiber housing guide 960.

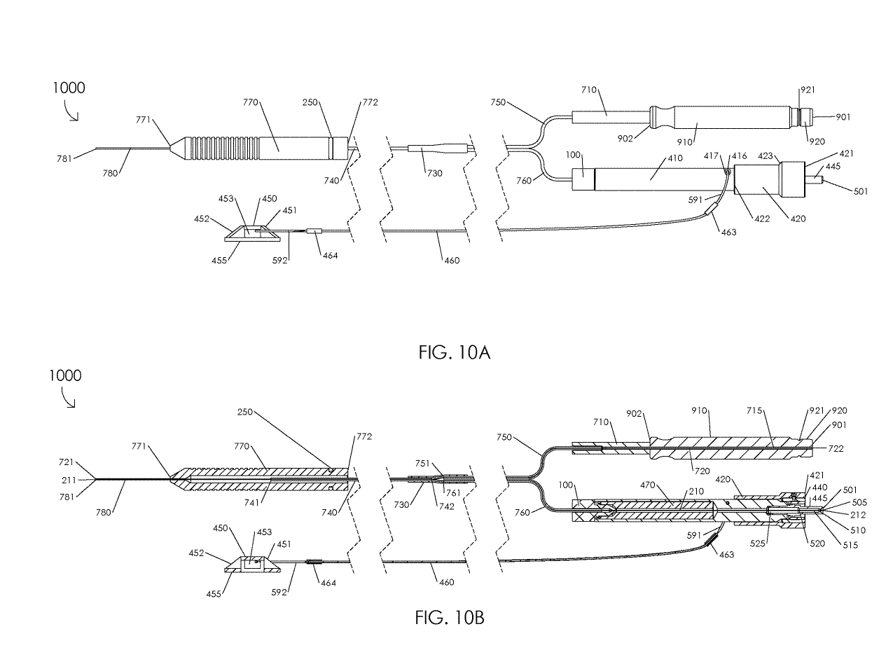

FIGS. 10A and 10B are schematic diagrams illustrating a single-use illuminated laser probe with optic fiber fixtures 1000. FIG. 10A illustrates a side view of a single-use illuminated laser probe with optic fiber fixtures 1000. FIG. 10B illustrates a cross-sectional view in a sagittal plane of a single-use illuminated laser probe with optic fiber fixtures 1000. Illustratively, a single-use illuminated laser probe with optic fiber fixtures 1000 may comprise an assembled single-use illuminated laser probe 800, an assembled optic fiber fixture 500, and an illumination optic fiber fixture 900. In one or more embodiments, a single-use illuminated laser probe with optic fiber fixtures 1000 may comprise a transitory connector 100 disposed in a portion of assembled optic fiber fixture 500 wherein optic fiber proximal end 212 is adjacent to assembled optic fiber fixture distal end 501. Illustratively, a single-use illuminated laser probe with optic fiber fixtures 1000 may comprise a transitory connector 100 disposed in a portion of assembled optic fiber fixture 500 wherein optic fiber proximal end 212 abuts assembled optic fiber fixture distal end 501. In one or more embodiments, a single-use illuminated laser probe with optic fiber fixtures 1000 may comprise a transitory connector 100 disposed in a portion of assembled optic fiber fixture 500 wherein optic fiber proximal end 212 is coplanar with assembled optic fiber fixture distal end 501. Illustratively, inserting a portion of optic fiber 210 into a portion of assembled optic fiber fixture 500 may be configured to ingress optic fiber proximal end 212 into transitory connector housing 550. In one or more embodiments, inserting a portion of optic fiber 210 into a portion of assembled optic fiber fixture 500 may be configured to ingress optic fiber proximal end 212 into transitory connector housing distal taper 545. Illustratively, transitory connector housing distal taper 545 may be configured to guide an ingress of optic fiber 210 into fixture facilitating sleeve inner bore 540. In one or more embodiments, inserting a portion of optic fiber 210 into a portion of assembled optic fiber fixture 500 may be configured to ingress optic fiber proximal end 212 into fixture facilitating sleeve inner bore 540. Illustratively, inserting a portion of optic fiber 210 into a portion of assembled optic fiber fixture 500 may be configured to ingress optic fiber proximal end 212 into fixture facilitating sleeve housing 535. In one or more embodiments, fixture facilitating sleeve housing 535 may be configured to guide an ingress of optic fiber 210 into fixture base inner bore 530. Illustratively, inserting a portion of optic fiber 210 into a portion of assembled optic fiber fixture 500 may be configured to ingress optic fiber proximal end 212 into fixture base inner bore 530. In one or more embodiments, inserting a portion of optic fiber 210 into a portion of assembled optic fiber fixture 500 may be configured to ingress optic fiber proximal end 212 into machine connector proximal taper 444. Illustratively, machine connector proximal taper 444 may be configured to guide an ingress of optic fiber 210 into machine connector proximal inner lumen 525. In one or more embodiments, inserting a portion of optic fiber 210 into a portion of assembled optic fiber fixture 500 may be configured to ingress optic fiber proximal end 212 into machine connector proximal inner lumen 525. Illustratively, inserting a portion of optic fiber 210 into a portion of assembled optic fiber fixture 500 may be configured to ingress optic fiber proximal end 212 into inner lumen proximal taper 520. In one or more embodiments, inner lumen proximal taper 520 may be configured to guide an ingress of optic fiber into machine connector distal inner lumen 515. Illustratively, inserting a portion of optic fiber 210 into a portion of assembled optic fiber fixture 500 may be configured to ingress optic fiber proximal end 212 into machine connector distal inner lumen 515. In one or more embodiments, inserting a portion of optic fiber 210 into a portion of assembled optic fiber fixture 500 may be configured to ingress optic fiber proximal end 212 into inner lumen distal taper 510. Illustratively, inner lumen distal taper 510 may be configured to guide an ingress of optic fiber 210 into optic fiber proximal end guide 505. In one or more embodiments, inserting a portion of optic fiber 210 into a portion of assembled optic fiber fixture 500 may be configured to ingress optic fiber proximal end 212 into optic fiber proximal end guide 505.

Illustratively, inserting transitory connector 100 into a portion of assembled optic fiber fixture 500 may be configured to ingress optic fiber 210 into optic fiber proximal end guide 505, e.g., inserting transitory connector 100 into a portion of assembled optic fiber fixture 500 may be configured to ingress optic fiber 210 into optic fiber proximal end guide 505 wherein optic fiber proximal end 212 is coplanar with assembled optic fiber fixture distal end 501. In one or more embodiments, inserting transitory connector distal end 101 into transitory connector housing distal taper 545 may be configured to ingress optic fiber 210 into optic fiber proximal end guide 505, e.g., inserting transitory connector distal end 101 into transitory connector housing distal taper 545 may be configured to ingress optic fiber 210 into optic fiber proximal end guide 505 wherein optic fiber proximal end 212 is coplanar with assembled optic fiber fixture distal end 501. Illustratively, inserting transitory connector nosecone base 120 into transitory connector housing 550 may be configured to ingress optic fiber 210 into optic fiber proximal end guide 505, e.g., inserting transitory connector nosecone base 120 into transitory connector housing 550 may be configured to ingress optic fiber 210 into optic fiber proximal end guide 505 wherein optic fiber proximal end 212 is coplanar with assembled optic fiber fixture distal end 501. In one or more embodiments, inserting raised helix 105 into transitory connector housing threading 560 may be configured to ingress optic fiber 210 into optic fiber proximal end guide 505, e.g., inserting raised helix 105 into transitory connector housing threading 560 may be configured to ingress optic fiber 210 into optic fiber proximal end guide 505 wherein optic fiber proximal end 212 is coplanar with assembled optic fiber fixture distal end 501. Illustratively, rotating raised helix 105 in transitory connector housing threading 560 may be configured to ingress optic fiber 210 into optic fiber proximal end guide 505, e.g., rotating raised helix 105 in transitory connector housing threading 560 may be configured to ingress optic fiber 210 into optic fiber proximal end guide 505 wherein optic fiber proximal end 212 is coplanar with assembled optic fiber fixture distal end 501. In one or more embodiments, rotating raised helix 105 in transitory connector housing threading 560 may be configured to fix transitory connector 100 in assembled optic fiber fixture 500, e.g., rotating raised helix 105 in transitory connector housing threading 560 may be configured to fix raised helix 105 in first transitory connector housing threading 560. For example, raised helix 105 may comprise a threaded fastener.

In one or more embodiments, a single-use illuminated laser probe with optic fiber fixtures 1000 may comprise an illumination optic fiber housing 715 disposed in a portion of illumination optic fiber fixture 900 wherein illumination optic fiber proximal end 722 is adjacent to illumination optic fiber fixture distal end 901. Illustratively, a single-use illuminated laser probe with optic fiber fixtures 1000 may comprise an illumination optic fiber housing 715 disposed in a portion of illumination optic fiber fixture 900 wherein illumination optic fiber proximal end 722 abuts illumination optic fiber fixture distal end 701. In one or more embodiments, a single-use illuminated laser probe with optic fiber fixtures 1000 may comprise an illumination optic fiber housing 715 disposed in a portion of illumination optic fiber fixture 900 wherein illumination optic fiber proximal end 722 is coplanar with illumination optic fiber fixture distal end 901.

Illustratively, inserting a portion of illumination optic fiber 720 into a portion of illumination optic fiber fixture 900 may be configured to ingress illumination optic fiber proximal end 722 into illumination optic fiber housing guide 960, e.g., inserting a portion of illumination optic fiber housing 715 into a portion of illumination optic fiber fixture 900 may be configured to ingress illumination optic fiber housing distal end 716 into illumination optic fiber housing guide 960. In one or more embodiments, illumination optic fiber housing guide 960 may be configured to guide an ingress of illumination optic fiber 720 into illumination optic fiber housing receptacle 930, e.g., illumination optic fiber housing guide 960 may be configured to guide an ingress of illumination optic fiber housing 715 into illumination optic fiber housing receptacle 930. Illustratively, inserting a portion of illumination optic fiber 720 into a portion of illumination optic fiber fixture 900 may be configured to ingress illumination optic fiber proximal end 722 into illumination optic fiber housing receptacle 930, e.g., inserting a portion of illumination optic fiber housing 715 into a portion of illumination optic fiber fixture 900 may be configured to ingress illumination optic fiber housing distal end 716 into illumination optic fiber housing receptacle 930.

In one or more embodiments, inserting a portion of illumination optic fiber 720 into a portion of illumination optic fiber fixture 900 until illumination optic fiber fixture interface 710 contacts illumination optic fiber fixture 900 may be configured to ingress illumination optic fiber 720 into illumination optic fiber fixture 900 wherein illumination optic fiber proximal end 722 is adjacent to illumination optic fiber fixture distal end 901, e.g., inserting a portion of illumination optic fiber 720 into a portion of illumination optic fiber fixture 900 until illumination optic fiber fixture interface distal end 711 contacts illumination optic fiber fixture proximal end 902 may be configured to ingress illumination optic fiber 720 into illumination optic fiber fixture 900 wherein illumination optic fiber proximal end 722 is adjacent to illumination optic fiber fixture distal end 901. Illustratively, inserting a portion of illumination optic fiber 720 into a portion of illumination optic fiber fixture 900 until illumination optic fiber fixture interface distal end 711 is adjacent to illumination optic fiber fixture proximal end 902 may be configured to ingress illumination optic fiber 720 into illumination optic fiber fixture 900 wherein illumination optic fiber proximal end 722 is adjacent to illumination optic fiber fixture distal end 901, e.g., inserting a portion of illumination optic fiber 720 into a portion of illumination optic fiber fixture 900 until illumination optic fiber fixture interface distal end 711 abuts illumination optic fiber fixture proximal end 902 may be configured to ingress illumination optic fiber 720 into illumination optic fiber fixture 900 wherein illumination optic fiber proximal end 722 is adjacent to illumination optic fiber fixture distal end 901. In one or more embodiments, inserting a portion of illumination optic fiber 720 into a portion of illumination optic fiber fixture 900 until illumination optic fiber fixture interface 710 contacts illumination optic fiber fixture 900 may be configured to ingress illumination optic fiber 720 into illumination optic fiber fixture 900 wherein illumination optic fiber proximal end 722 is coplanar with illumination optic fiber fixture distal end 901, e.g., inserting a portion of illumination optic fiber 720 into a portion of illumination optic fiber fixture 900 until illumination optic fiber fixture interface distal end 711 contacts illumination optic fiber fixture proximal end 902 may be configured to ingress illumination optic fiber 720 into illumination optic fiber fixture 900 wherein illumination optic fiber proximal end 722 is coplanar with illumination optic fiber fixture distal end 901. Illustratively, inserting a portion of illumination optic fiber 720 into a portion of illumination optic fiber fixture 900 until illumination optic fiber fixture interface distal end 711 is adjacent to illumination optic fiber fixture proximal end 902 may be configured to ingress illumination optic fiber 720 into illumination optic fiber fixture 900 wherein illumination optic fiber proximal end 722 is coplanar with illumination optic fiber fixture distal end 901, e.g., inserting a portion of illumination optic fiber 720 into a portion of illumination optic fiber fixture 900 until illumination optic fiber fixture interface distal end 711 abuts illumination optic fiber fixture proximal end 902 may be configured to ingress illumination optic fiber 720 into illumination optic fiber fixture 900 wherein illumination optic fiber proximal end 722 is coplanar with illumination optic fiber fixture distal end 901.

Illustratively, a user may perform an illuminated photocoagulation procedure with a single-use illuminated laser probe with optic fiber fixtures 1000. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, a user may connect illumination optic fiber fixture 900 to an illumination machine. Illustratively, a user may energize the illumination machine to deliver illumination light into illumination optic fiber proximal end 722, through illumination optic fiber 720, out from illumination optic fiber distal end 721, and onto a surgical target site. In one or more embodiments, illumination optic fiber fixture 900 may be a reusable medical device sold non-sterile and sterilized by a user in a medical autoclave. Illustratively, assembled single-use illuminated laser probe 800 may be a single-use medical device sold sterile and discarded after use. In one or more embodiments, optic fiber 210 may be manufactured from glass, e.g., optic fiber 210 may be manufactured from silica. Illustratively, optic fiber 210 may comprise a plurality of optic fibers 210. In one or more embodiments, illumination optic fiber 720 may comprise a plurality of illumination optic fibers 720. Illustratively, illumination optic fiber 720 may comprise one or more optic fibers manufactured from plastic, e.g., illumination optic fiber 720 may comprise one or more optic fibers manufactured from Polymethyl Methacrylate Resin, Polystyrene, etc. In one or more embodiments, illumination optic fiber 720 may comprise one or more optic fibers having a cladding material, e.g., illumination optic fiber 720 may comprise one or more optic fibers having a cladding material manufactured from a fluorinated polymer, a silicone resin, etc. Illustratively, illumination optic fiber 720 may comprise one or more optic fibers having a step index refractive index profile. In one or more embodiments, illumination optic fiber 720 may comprise one or more multi-mode optic fibers, one or more single-mode optic fibers, etc. In one or more embodiments, illumination optic fiber 720 may comprise one or more optic fibers having a core refractive index in a range of 1.3 to 1.8, e.g., illumination optic fiber 720 may comprise one or more optic fibers having a core refractive index of 1.49. Illustratively, illumination optic fiber 720 may comprise one or more optic fibers having a core refractive index of less than 1.3 or greater than 1.8. In one or more embodiments, illumination optic fiber 720 may comprise one or more optic fibers having a numerical aperture in a range of 0.3 to 0.8, e.g., illumination optic fiber 720 may comprise one or more optic fibers having a numerical aperture of 0.5. In one or more embodiments, illumination optic fiber 720 may comprise one or more optic fibers having a numerical aperture of less than 0.3 or greater than 0.8. Illustratively, illumination optic fiber 720 may comprise one or more optic fibers having a core diameter in a range of 85 to 285 micrometers, e.g., illumination optic fiber 720 may comprise one or more optic fibers having a core diameter of 135 micrometers. In one or more embodiments, illumination optic fiber 720 may comprise one or more optic fibers having a core diameter of less than 85 micrometers or greater than 285 micrometers. Illustratively, illumination optic fiber 720 may comprise one or more optic fibers having an overall diameter in a range of 100 to 300 micrometers, e.g., illumination optic fiber 720 may comprise one or more optic fiber having an overall diameter of 200 micrometers. In one or more embodiments, illumination optic fiber 720 may comprise one or more optic fibers having an overall diameter of less than 100 or greater than 300 micrometers.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a laser probe, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a handle having a handle distal end and a handle proximal end;
a hypodermic tube having a hypodermic tube distal end and a hypodermic tube proximal end, the hypodermic tube disposed in a hypodermic tube housing of the handle wherein the hypodermic tube distal end extends out from the handle distal end;
a transitory connector having a transitory connector distal end and a transitory connector proximal end;
an illumination optic fiber housing having an illumination optic fiber housing distal end and an illumination optic fiber housing proximal end;
an optic fiber having an optic fiber distal end and an optic fiber proximal end, the optic fiber disposed in the handle, the hypodermic tube, and the transitory connector wherein the optic fiber distal end is adjacent to the hypodermic tube distal end and wherein the optic fiber proximal end extends out from the transitory connector distal end; and
an illumination optic fiber having an illumination optic fiber distal end and an illumination optic fiber proximal end, the illumination optic fiber disposed in the handle, the hypodermic tube, and the illumination optic fiber housing wherein the illumination optic fiber distal end is adjacent to the hypodermic tube distal end and wherein the illumination optic fiber proximal end is adjacent to the illumination optic fiber housing distal end.

2. The instrument of claim 1 further comprising:
an illumination optic fiber fixture interface having an illumination optic fiber fixture interface distal end and an illumination optic fiber fixture interface proximal end, the illumination optic fiber housing disposed in the illumination optic fiber fixture interface wherein the illumination optic fiber housing distal end extends out from the illumination optic fiber fixture interface distal end.

3. The instrument of claim 1 further comprising:
an illumination optic fiber fixture having an illumination optic fiber fixture distal end and an illumination optic fiber fixture proximal end wherein the illumination optic fiber fixture is configured to interface with an illumination machine.

4. The instrument of claim 1 further comprising:
an optic fiber fixture having an optic fiber fixture distal end and an optic fiber fixture proximal end wherein the optic fiber fixture is configured to interface with a laser machine.

5. The instrument of claim 4 further comprising:
a fixture base of the optic fiber fixture having a fixture base distal end and a fixture base proximal end.

6. The instrument of claim 5 further comprising:
a fixture facilitating sleeve having a fixture facilitating sleeve distal end and a fixture facilitating sleeve proximal end, the fixture facilitating sleeve disposed in the fixture base.

7. The instrument of claim 6 wherein a coefficient of friction between a portion of the optic fiber and a portion of the fixture facilitating sleeve is in a range of 0.011 to 0.36.

8. The instrument of claim 6 wherein a coefficient of friction between a portion of the optic fiber and a portion of the fixture facilitating sleeve is less than 0.011.

9. The instrument of claim 6 wherein the fixture facilitating sleeve is manufactured from a self-lubricating thermoplastic.

10. The instrument of claim 6 wherein the fixture facilitating sleeve is manufactured from a fluorocarbon.

11. The instrument of claim 6 wherein the fixture facilitating sleeve is manufactured from a turcite.

12. The instrument of claim 6 further comprising:
a transitory connector housing of the fixture facilitating sleeve wherein disposing the transitory connector in the transitory connector housing is configured to ingress the optic fiber proximal end through the optic fiber fixture wherein the optic fiber proximal end is adjacent to the optic fiber fixture distal end.

13. The instrument of claim 1 wherein the optic fiber is fixed in the hypodermic tube.

14. The instrument of claim 1 wherein the hypodermic tube is fixed in the handle.

15. The instrument of claim 1 wherein the optic fiber is fixed in the transitory connector.

16. The instrument of claim 1 wherein the illumination optic fiber is fixed in the hypodermic tube.

17. The instrument of claim 1 wherein the illumination optic fiber is fixed in the illumination optic fiber housing.

18. The instrument of claim 1 further comprising:
a coupling sleeve.

19. The instrument of claim 1 further comprising:
a dual fiber housing.

20. The instrument of claim 1 further comprising:
a jacketing; and
an illumination jacketing.
